(19) United States
(12) Patent Application Publication
(10) Pub. No.: US 2002/0049996 A1
(43) Pub. Date: Apr. 25, 2002

(54) Nucleic acid and amino acid sequences encoding a de novo DNA methyltransferase

(76) Inventors: Shawn M. Kaeppler, Oregon, WI (US); Nathan M. Springer, Northfield, MN (US); Michael G. Muszynski, Clive, IA (US)

Correspondence Address: ROCKEY, MILNAMOW & KATZ, LTD., Two Prudential Plaza, 180 North Stetson Avenue, Suite 4700, Chicago, IL, 60601, US

(21) Appl. No.: 09/767,536

(22) Filed: Jan. 23, 2001

Related U.S. Application Data
(60) Provisional application No. 60/177,753, filed on Jan. 24, 2000.

(51) Int. Cl. A01H 5/00; C12N 15/82

(52) U.S. Cl. 800/298; 536/23.2; 536/23.6; 435/320.1; 435/252.3; 800/278

(57) ABSTRACT

The present invention provides nucleic acids encoding polypeptides which encode a de novo DNA methyltransferase. These nucleic acids can be used to stabilize transgene expression in transgenic plants, to alter the yield or biochemical qualities of plants to silencing targeted genes in plants in vivo.

### PRIORITY INFORMATION

This applications claims priority from U.S. Ser. No. 60/177,753 filed Jan. 24, 2000.

This invention was made with United States government support awarded by the following agencies: USDA 99-CRHF-0-6055. The United States has certain rights in the invention.

### FIELD OF THE INVENTION

The present invention relates to nucleic acid and amino acid sequences which encode a de novo DNA methyltransferase. The present invention further relates to methods of using the nucleic acid and amino acid sequences described herein to stabilize transgene expression in transgenic plants, to alter the yield or biochemical qualities of plants and to silence targeted genes in plants in vivo.

### BACKGROUND OF THE INVENTION

The information content of a primary DNA sequence can be enhanced by the addition of a methyl group to the ring structure of cytosine or adenine residues (Finnegan, E. J., et al., Annu. Rev. Plant Physiol. Plant Mol. Biol. 49:223-47 (1998)). The chemical modification of DNA is known to affect protein-DNA interactions. Specifically, in prokaryotes, methylation of DNA prevents cleavage by the cognate restriction endonucleases. Id. In higher eukaryotes, cytosine methylation can inhibit binding of regulatory proteins and methylation of promoter and coding sequences of genes can repress transcription, both in vitro and in vivo. Id. Methylation of DNA has been implicated in the timing of DNA replication, in determination of chromatin structure, in increasing mutation frequency, as a causal agent for some human diseases, and as a basis for epigenetic phenomena. Id.

Eukaryotic genomes are not methylated uniformly, but instead contain specific methylated regions, with other domains remaining umnethylated (Martienssen, R. A., et al., Current Opinion in Genetics and Development, 5:234-242 (1995)). The enzymes that transfer methyl groups to the cytosine ring are cytosine-5-methyltransferases (hereinafter referred to as “DNA methyltransferases”) and have been characterized from a number of eukaroytes. All characterized eukaryotic DNA methyltransferases exhibit little primary sequence specificity in vitro other than the short canonical symmetrical sites methylated which are CpG in animals, and CpG and CpNpG in plants (where N stands for any nucleotide). Mammalian and plant genomes contain methylation-free GC-rich zones, or CpG islands, which are frequently associated with the 5′ regions of housekeeping genes. Id.

In plants, DNA methylation is necessary for normal development. For example, Arabidopsis having reduced levels of DNA methylation demonstrate a range of abnormalities, including loss of apical dominance, reduced stature, altered leaf size and shape, reduced root length, homeotic transformation of floral organs and reduced fertility (Finnegan, E. J., et al., Annu. Rev. Plant Physiol. Plant Mol. Biol. 49:223-47 (1998)). Moreover, Arabidopsis plants in which methylation had been reduced by at least 70% became infertile after four to five generations of selfing. Id. A comparable reduction in DNA methylation is embryo lethal in mammals. Id.

Two classes of DNA methyltransferase enzymes have been cloned in plants (Finnegan, E. J., et al., Annu. Rev. Plant Physiol. Plant Mol. Biol. 49:223-47 (1998))—class I and class II. Class I enzymes include MetI and MetlI from Arabidopsis (Finnegan et al. Nucleic Acids Res., 21(10):2383-2388 (1993); Nebendahl, et al., Gene 157(1-2):269-272 (1995)), Met1-5 and Met2-21 from carrot (Bemacchia, G et al., Plant Physiol. 116:446-446 (1998)), C-5 MTase from tomato (Bemacchia, G et al. Plant J, 13(3):317-330 (1998)), and C-5 MTase from pea (Pradhan et al., Nucleic Acids Res., 26(5):1214-1222 (1998)). Class II sequences have been detected in many species with a defining characteristic of the presence of an embedded chromodomain (Rose et al., Nucleic Acids Res., 26(7):1628-1635 (1998)). The only full-length class II sequence is CmtI from Arabidopsis (Genbank #AF039364).

Class I enzymes are homologous to dnmt1 from mice (Bestor, T., et al., EMBO J, 11(7):2611-2617 (1988)), the first cloned DNA methyltransferase. A knockout of dnmt1 in mice resulted in lethality during embryogenesis (Li et al., Cell, 69(6):915-926 (1992)). Dnmt1 has been used as a model for all class I enzymes though it has not been proven whether this is appropriate in plant systems. Antisense expression of MetI in Arabidopsis resulted in numerous developmental abnormalities (Finnegan et al., Proc. Natl. Acad. Sci. U.S.A., 93(16):8449-8454 (1996)). Class I enzymes are thought to function as maintenance enzymes, though proteolytic cleavage could create de novo enzymes (Bestor, T. H., EMBO J, 11 (7):2611-2617 (1992)). CpG activity has been shown for dnmt1 in mice and humans. In peas it was found that pea C-5 MTase expressed in baculovirus displayed both CpG and CpNpG activity (Pradhan et al., Nucleic Acids Res., 26(5):1214-1222 (1998)). In general, class I enzymes have a high level of expression in tissues that are actively dividing and are expressed at lower levels or silent in mature tissues.

There is little known regarding the function of class II enzymes. CmtI was detected as an Arabidopsis genomic sequence based on sequence homology to other methyltransferases. The C-terminal region contains the conserved methyltransferase domains and a chromodomain. The N-terminal region is much shorter than the N-terminal region of class I enzymes. Several commonly used ecotypes of Arabidopsis contain an allele of Cmt1 which is interrupted by a transposon insertion. These Cmt1 knockouts do not have any detectable phenotype. No other research has been published on the function of class II enzymes. Cmt1 is expressed only in floral tissues at very low levels. Degenerate PCR has been used to show the presence of Cmt1 homologs in a number of other plant species (Rose et al., Nucleic Acids Res., 26(7):1628-1635 (1998)). In addition to finding homologs in other species, two sequences with similarity to Cmt1, Cmt2 and Cmt3, were identified in the Arabidopsis.

DNA methylation provides a mechanism for the mitotic propagation of epigenetic states. Epigenetic lineage-dependent patterns of gene expression have been studied the most in the germline and in somatic cell lineages in multicellular eukaryotes (Martienssen, R. A., et al., Curr. Opin. Genet. and Develop., 5:234-242 (1995)). For example, in mice, the parentally imprinted genes H19 and Igf2r are expressed in the embryo only when they are inherited via the female gamete. Id. In contrast, the Igf2 gene is expressed only when inherited via the male gamete. Id. The human homologs of the Igf2 and H19 genes are linked and parentally imprinted as in the mouse. Id. Parental uniparental disomy for this chromosomal region (11p15) is associated with Beckwith-Wiedemann syndrome, which is believed to result from overexpression of Igf2. Id. In addition to overgrowth of certain organs, Beckwith-Wiedemann syndrome patients have a 700-fold predisposition to Wilms' tumor, and loss of heterozygosity in this region is found in many other tumors as well. Id. It has also been shown that 60-70% of Wilms' tumor patients have biallelic expression of Igf2, H19, or both in tumor tissue, resulting from loss of imprinting rather than loss of heterozygosity. Id.

In plants, epigenetic changes in gene expression are considered to be easier to observe than in animals since there is little cell migration and clonal lineages stay together. Id. Moreover, because in plants the germline arises relatively late in development, many somatically variegated phenotypes can be followed into the next generation and are heritable to greater or lesser extents. Id. Parental imprinting of gene expression was first observed in plants at the R locus in maize. Id. Certain alleles condition a mottled phenotype in the alerone layer of the extra-embryonic endosperm when inherited paternally, but cause a fully colored phenotype when inherited maternally. Id. Genetic studies of modifier loci have revealed that it is the maternally inherited R allele that is imprinted to a high level of expression. Id. High levels of R expression correlate with demethylation of sites in the transcribed region in the maternally inherited allele. Id.

Plants transformed with additional copies of endogenous genes or with multiple copies of a foreign or exogenous gene (these endogenous and exogenous genes are often referred to as “transgenes”) frequently display epigenetic inactivation. This phenomenon is known as “gene silencing” or “co-suppression”. There are two types of “gene silencing” or “co-suppression”. The first is “transcriptional silencing”. In “transcriptional silencing”, RNA production from the introduced transgene is repressed. The second type of “gene silencing” is “posttranscriptional silencing”. In “posttranscriptional silencing”, transcripts do not accumulate in the cytoplasm even though transcription rates are comparable with or are higher than those in cells where transcripts do accumulate.

Transcriptional silencing is associated with transgene methylation, particularly in the promoter (Finnegan, E. J., et al., Annu. Rev. Plant Physiol. Plant Mol. Biol. 49:223-47 (1998)). Posttranscriptional silencing, which affects both transgenes and homologous endogeneous genes, is also associated with transgene methylation, but within the coding sequence rather than the promoter. Id. It is believed that both forms of gene silencing reflect normal, cellular defenses against invading or mobile DNAs. Id.

Currently, two classes of methyltransferase genes have been cloned in maize. The class I clone homolog is referred to as Zmet1 and the class II homolog Zmet2. The Zmet1 is a class I enzyme that was cloned by Paula Olhoft and Ron Phillips at the University of Minnesota. The full-length sequence and function of the zmet3 de novo methyltransferase gene has now been characterized and is described herein and is the subject of the present invention.

### SUMMARY OF THE INVENTION

The present invention relates to an isolated and purified Zea mays zmet3 methyltransferase polynucleotide. The Zea mays zmet3 methyltransferase polynucleotide hybridizes to SEQ ID NO:1 under stringent conditions. The polynucleotide of the present invention is unique in that it displays rearranged DNA catalytic motifs.

The amino acid encoded by the zmet3 methyltransferase polynucleotide is shown in SEQ ID NO:2 and contains the hereinbefore described rearranged catalytic domains.

The present invention further provides for recombinant expression cassettes containing a promoter sequence operably linked to the isolated and purified Zea mays zmet3 methyltransferase polynucleotide. A polyadenylation signal can also be operably linked to the the isolated and purified Zea mays zmet3 methyltransferase polynucleotide. The promoter can be a constitutive or a tissue specific promoter. Bacterial cells, plant cells, plants and seeds can then be transformed with this recombinant expression cassette. Monocotyledonous or dicotyledonous plant cells, plants and seeds can be transformed with this expression cassette. Plants which can be transformed with the recombinant expression cassette of the present invention include, but are not limited to, Zea mays, Oryza sativa, Secale cereale, Triticum aestivum, Daucus carota, Brassica oleracea, Cucumis melo, Cucumis sativus, Latuca sativa, Solanum tubersoum, Lycopersicon esculentum, Phaseolus vulgaris, Brassica napus, etc.

The present invention further provides methods of reducing or altering methyltransferase activity in a transgenic plant in order to increase transgene expression stability and/or to improve the yield or biochemical qualities of a plant as well as a method of silencing targeted genes in a plant in vivo. Each of these methods comprise introducing into an appropriate plant, which can be either a transgenic or a non-transgenic plant, a recombinant expression cassette comprising an appropriate plant promoter, such as a tissue-specific promoter, operably linked to the isolated and purified Zea mays zmet3 methyltransferase polynucleotide in either the sense or antisense direction.

Definitions

Units, prefixes, and symbols can be denoted in the SI accepted form. Numeric ranges are inclusive of the numbers defining the range. Unless otherwise indicated, nucleic acids are written left to right in 5′ to 3′ orientation, respectively. The headings provided herein are not limitations of the various aspects or embodiments of the invention which can be had by reference to the specification as a whole. Accordingly, the terms defined immediately below are more fully defined by reference to the specification as a whole.

As used herein, the term “plant” includes reference to whole plants, plant organs (e.g., leaves, stems, roots, etc.), seeds and plant cells and progeny thereof. The class of plants which can be used in the methods of the present invention are generally as broad as the class of higher plants amenable to transformation techniques, including both monocotyledonous and dicotyledonous plants.

As used herein, “heterologous” when used to describe nucleic acids or polypeptides refers to nucleic acids or polypeptides that originate from a foreign species, or, if from the same species, are substantially modified from their original form. For example, a promoter operably linked to a heterologous structural gene is from a species different from that from which the structural gene was derived, or, if from the same species, one or both are substantially modified from their original form.

A polynucleotide or polypeptide is “exogenous to” an individual plant when it is introduced into a plant by any means other than by a sexual cross. Examples of means by which this can be accomplished are described below, and include Agrobacterium-mediated transformnation, biolistic methods, electroporation, and the like. Such a plant containing the exogenous nucleic acid is referred to herein as an R₁ generation transgenic plant. Transgenic plants which arise from sexual cross or by selfing are descendants of such a plant.

As used herein, “zmet3 methyltransferase gene” or “zmet3 methyltransferase polynucleotide” refers to a polynucleotide encoding zmet3 methyltransferase and which hybridizes under stringent conditions and/or has at least 60% sequence identity at the deduced amino acid level to the exemplified sequences provided herein. The zmet3 polypeptide encoded by the zmet3 methyltransferase gene has at least 55% or 60% sequence identity, typically at least 65% sequence identity, preferably at least 70% sequence identity, often at least 75% sequence identity, more preferably at least 80% sequence identity, and most preferably at least 90% sequence identity at the deduced amino acid level relative to the exemplary zmet3 methyltransferase sequences provided herein.

As used herein, “zmet3 methyltransferase polynucleotide” includes reference to a contiguous sequence from a zmet3 methyltransferase gene of at least 1810 nucleotides in length. In some embodiments the polynucleotide is preferably at least 2119 nucleotides in length and more preferably at least 2378 nucleotides in length.

As used herein, “isolated” includes reference to material which is substantially or essentially free from components which normally accompany or interact with it as found in its naturally occurring environment. The isolated material optionally comprises material not found with the material in its natural environment.

As used herein, “nucleic acid” includes reference to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. Unless otherwise indicated, a particular nucleic acid sequence includes the complementary sequence thereof.

As used herein, “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to joint two protein coding regions, contiguous and in the same reading frame.

In the expression of transgenes, one of ordinary skill in the art will recognize that the inserted polynucleotide sequence need not be identical and may be “substantially identical” to a sequence of the gene from which it was derived. As explained below, these variants are specifically covered by this term.

In the case where the inserted polynucleotide sequence is transcribed and translated to produce a functional zmet3 methyltransferase polypeptide, one of ordinary skill in the art will recognize that because of codon degeneracy, a number of polynucleotide sequences will encode the same polypeptide. These variants are specifically covered by the term “zmet3 methyltransferase polynucleotide sequence”. In addition, the term specifically includes those full length sequences substantially identical (determined as described below) with a zmet3 methyltransferase gene sequence which encode proteins that retain the function of the zmet3 methyltransferase. Thus, in the case of the zmet3 methyltransferase genes disclosed herein, the term includes variant polynucleotide sequences which have substantial identity with the sequences disclosed herein and which encode proteins capable of reducing or regulating DNA methylation in a transgenic plant for various purposes as well as silencing target genes in a plant using the polynucleotide sequences described herein.

Two polynucleotides or polypeptides are said to be “identical” if the sequence of nucleotides or amino acid residues, respectively, in the two sequences is the same when aligned for maximum correspondence as described below. The term “complementary to” is used herein to mean that the complementary sequence is identical to all or a specified contiguous portion of a reference polynucleotide sequence. Sequence comparisons between two (or more) polynucleotides or polypeptides are typically performed by comparing sequences of two optimally aligned sequences over a segment or “comparison window” to identify and compare local regions of sequence similarity. Optimal alignment of sequences for comparison may be conducted by the local homology algorithm of Smith and Waterman, Ad. App. Math. 2: 482 (1981), by the homology alignment algorithm of Neddleman and Wunsch, J. Mol Biol. 48:443 (1970), by the search for similarity method of Pearson and Lipman, Proc. Natl. Acad. Sci. (U.S.A.) 85:2444 (1988), by computerized implementation of these algorithms (GAP, BESTFIT, FASTA, and TFASTA in the Wisconsin Genetics Software Package, Genetics Computer Group (GCG), 575 Science Dr., Madison, Wis.), or by inspection.

“Percentage of sequence identity” is determined by comparing two optimally aligned sequences over a comparison window, where the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 55% or 60% sequence identity, generally at least 65%, preferably at least 70%, often at least 75%, more preferably at least 80% and most preferably at least 90%, compared to a reference sequence using the programs described above (preferably BESTFIT) using standard parameters. One of ordinary skill in the art will recognize that these values can be appropriately adjusted to determine corresponding identity of proteins encoded by two nucleotide sequences by taking into account codon degeneracy, amino acid sequences for those purposes normally means sequence identity of at least 55% or 60%, preferably at least 70%, more preferably at least 80%, and most preferably at least 95%. Polypeptides having “sequence similarity” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

Another indication that nucleotide sequences are substantially identical is if two molecules hybridize to each other under appropriate conditions. Appropriate conditions can be high or low stringency and will be different in different circumstances. Generally, stringent conditions are selected to be about 5° C. to about 20° C. lower than the thermal melting point (Tₘ) for the specific sequence at a defined ionic strength and pH. The Tₘ is the temperature (under defined ionic strength and pH 0) at which 50% of the target sequence hybridizes to a perfectly matched probe. Typically, stringent wash conditions are those in which the salt concentration is about 0.22 molar at pH 7 and the temperature is at least about 50° C. However, nucleic acids which do not hybridize to each other under stringent conditions are still substantially identical if the polypeptides which they encode are substantially identical. This may occur, e.g., when a copy of a nucleic acid is created using the maximum codon degeneracy permitted by the genetic code.

Nucleic acids of the present invention can be identified from a cDNA or genomic library prepared according to standard procedures and the nucleic acids disclosed here used as a probe. For example, stringent hybridization conditions will typically include at least one low stringency wash using 0.3 molar salt (e.g., 2×SSC) at 65° C. The washes are preferably followed by one or more subsequent washes using 0.03 molar salt (e.g., 0.2×SSC) at 50° C., usually 60° C., or more usually 65° C. Nucleic acid probes used to isolate the nucleic acids are preferably at least 100 nucleotides in length.

As used herein, a homologue of a particular zmet3 methyltransferase gene is a second gene (either in the same species or in a different species) which encodes a protein having an amino acid sequence having at least 50% identity or 75% similarity to (determined as described above) to a polypeptide sequence in the first gene product.

As used herein, “nucleotide binding site” or “nucleotide binding domain” includes reference to a region consisting of kinase-1a, kinase 2, and kinase 3a motifs, which participates in ATP/GTP-binding. Such motifs are described for instance in Yu et al., Proc. Acad. Sci USA 93:11751-11756 (1996); Mindrinos, et al., Cell 78:1089-1099 and Shen et al., FEBS, 335:380-385 (1993).

As used herein, “tissue-specific promoter” includes reference to a promoter in which expression of an operably linked gene is limited to a particular tissue or tissues.

As used herein “recombinant” includes reference to a cell, or nucleic acid, or vector, that has been modified by the introduction of a heterologous nucleic acid or the alteration of a native nucleic acid to a form not native to that cell, or that the cell is derived from a cell so modified. For example, recombinant cells express genes that are not found within the native (non-recombinant) form of the cell or express native genes that are otherwise abnormally expressed, under expressed or not expressed at all.

As used herein, a “recombinant expression cassette” is a nucleic acid construct, generated recombinantly or synthetically, with a series of specified nucleic acid elements which permit transcription of a particular nucleic acid in a target cell. The expression vector can be part of a plasmid, virus, or nucleic acid fragment. Typically, the recombinant expression cassette portion of the expression vector includes a nucleic acid to be transcribed, and a promoter.

As used herein, “transgenic plant” includes reference to a plant modified by introduction of a heterologous polynucleotide. Generally, the heterologous polynucleotide is a zmet3 methyltransferase structural or regulatory gene or subsequences thereof.

As used herein, “hybridization complex” includes reference to a duplex nucleic acid sequence formed by selective hybridization of two single-stranded nucleic acids with each other.

As used herein, “amplified” includes reference to an increase in the molarity of a specified sequence. Amplification methods include the PCR, the ligase chain reaction (hereinafter “LCR”), the transcription-based amplification system (hereinafter “TAS”), the self-sustained sequence replication system (hereinafter “SSR”). A wide variety of cloning methods, host cells, and in vitro amplification methodologies are well-known to persons of ordinary skill in the art

As used herein, “nucleic acid sample” includes reference to a specimen suspected of comprising zmet3 methyltransferase genes.

### SEQUENCE LISTINGS

The present application also contains a sequence listing that contains eight (8) sequences. The sequence listing contains nucleotide sequences and amino acid sequences. For the nucleotide sequences, the base pairs are represented by the following base codes:

| Symbol | Meaning |
|---|---|
| A | A; adenine |
| C | C; cytosine |
| G | G; guanine |
| T | T; thymine |
| U | U; uracil |
| M | A or C |
| R | A or G |
| W | A or T/U |
| S | C or G |
| Y | C or T/U |
| K | G or T/U |
| V | A or C or G; not T/U |
| H | A or C or T/U; not G |
| D | A or G or T/U; not C |
| B | C or G or T/U; not A |
| N | (A or C or G or T/U) |

The amino acids shown in the application are in the L-form and are represented by the following amino acid-three letter abbreviations:

| Abbreviation | Amino acid name |
|---|---|
| Ala | L-Alanine |
| Arg | L-Arginine |
| Asn | L-Asparagine |
| Asp | L-Aspartic Acid |
| Asx | L-Aspartic Acid or Asparagine |
| Cys | L-Cysteine |
| Glu | L-Glutamic Acid |
| Gln | L-Glutamine |
| Glx | L-Glutamine or Glutamic Acid |
| Gly | L-Glycine |
| His | L-Histidine |
| Ile | L-Isoleucine |
| Leu | L-Leucine |
| Lys | L-Lysine |
| Met | L-Methionine |
| Phe | L-Phenylalanine |
| Pro | L-Proline |
| Ser | L-Serine |
| Thr | L-Threonine |
| Trp | L-Tryptophan |
| Tyr | L-Tyrosine |
| Val | L-Valine |
| Xaa | L-Unknown or other |

### BRIEF DESCRIPTION OF THE DRAWINGS

FIG. 1 shows the polynucleotide sequence of the zmet3 methyltransferase gene.

FIG. 2 shows the amino acid sequence of the zmet3 methyltransferase gene.

FIG. 3 shows a schematic diagram of the domain structures of mouse Dnmt3b and zmet3 drawn to scale. Shaded boxes show the different motifs present in these proteins including the PWWP and cysteine rich (hereinafter “C-rich”) motifs present in Dnmt3b and the ubiquitin associated (hereinafter “UBA”) domains present in zmet3. Roman numerals denote the motifs of the methyltransferase catalytic domains.

FIG. 4 shows the alignment of zmet3 from Zea mays and the methyltransferase catalytic domains of mouse Dnmt3b (GenBank Accession AF068628) and Danio rerio Zmet3 (Danmt3; GenBank Accession AF135438). Pound symbols show the point of rearrangement of the plant proteins relative to the animal proteins. The numbering of the animal methyltransferases begins at amino acid 581 for Dnmt3b and 558 for Danmt3. Conserved catalytic motifs I-VI and IX-X are indicated. Asterisks denote conserved amino acids present in each motif.

FIG. 5 shows a table containing the percentage identity between either the C terminal domains or the N terminal domains of the proteins shown in FIG. 4.

### DESCRIPTION OF THE PREFERRED EMBODIMENTS

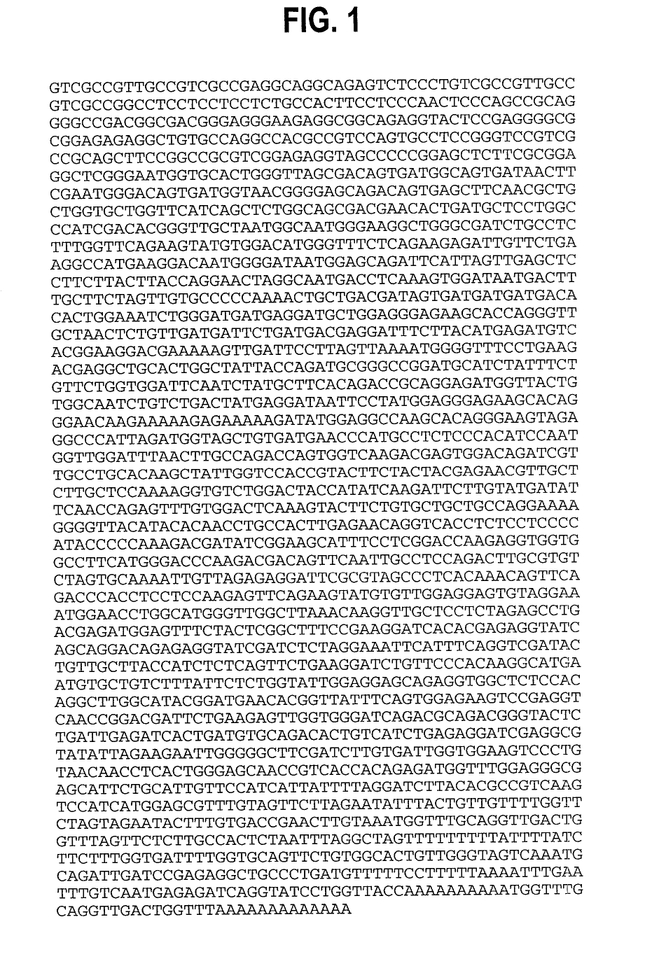

The present invention relates to a zmet3 methyltransferase gene. The zmet3 methyltransferase gene (shown in SEQ ID NO:1 and FIG. 1) encodes a de novo methyltransferase gene which controls DNA methylation. Nucleic acid sequences from the zmet3 methyltransferase gene can be used to reduce or alter the level of DNA methylation in a plant. In addition, the nucleic acid sequences described herein can be used to methylate a target gene in a plant in vivo to “silence” or “knock-out” said gene.

The present invention is applicable to a broad range of types of plants, including, but not limited to, Zea mays, Oryza sativa, Secale cereale, Triticum aestivum, Daucus carota, Brassica oleracea, Cucumis melo, Cucumis sativus, Latuca sativa, Solanum tubersoum, Lycopersicon esculentum, Phaseolus vulgaris, and Brassica napus.

The nucleic acids of the present invention can be used in marker-aided selection. Marker-aided selection does not require the complete sequence of the gene or precise knowledge of which sequence confers which specificity. Instead, partial sequences can be used as hybridization probes or as the basis for oligonucleotide primers to amplify by PCR or other methods to follow the segregation of chromosome segments containing the zmet3 methyltransferase gene in plants. Because the zmet3 methyltransferase marker is the gene itself, there can be negligible recombination between the marker and the methylated phenotype. Thus, the polynucleotides of the present invention can be used to provide an optimal means to DNA fingerprint de novo DNA methyltransferases in other cultivars and wild germplasm. This can be used to indicate if other germplasm accessions and cultivars carry the same zmet3 methyltransferase genes.

Preparation of Nucleic Acids of the Present Invention

Generally, the nomenclature and the laboratory procedures involved with recombinant DNA technology described below are those well known and commonly employed by those of ordinary skill in the art. Standard techniques are used for cloning, DNA and RNA isolation, amplification and purification. Generally, enzymatic reactions involving DNA ligase, DNA polymerase, restriction endonucleases and the like are performed according to the manufacturer's specifications. These techniques and various other techniques are generally performed according to Sambrook et al., Molecular Cloning—A Laboratory Manual, Cold Spring Harbor Laboratory, Cold Spring Harbor, N.Y. (1989).

The isolation of zmet3 methyltransferase genes may be accomplished by a number of techniques. For instance, oligonucleotide probes based on the sequences disclosed herein can be used to identify the desired gene in a cDNA or genomic DNA library. To construct genomic libraries, large segments of genomic DNA are generated by random fragmentation, e.g. using restriction endonucleases, and are ligated with vector DNA to form concatemers that can be packaged into the appropriate vector. To prepare a cDNA library, mRNA is isolated from the desired organ of a particular plant, such as shoots from Zea mays, and a cDNA library which contains the zmet3 methyltransferase gene transcript is prepared from the mRNA. Alternatively, cDNA may be prepared from mRNA extracted from other tissues in which the zmet3 methyltransferase gene or homologs are expressed.

The cDNA or genomic library can then be screened using a probe based upon the sequence of a cloned zmet3 methyltransferase gene such as the zmet3 methyltransferase gene disclosed herein. Probes may be used to hybridize with genomic DNA or cDNA sequences to isolate homologous genes in the same or different plant species.

Those of ordinary skill in the art will appreciate that various degrees of stringency of hybridization can be employed in the assay and either the hybridization or the wash medium can be stringent. As the conditions for hybridization become more stringent, there is a greater degree of complementarity required between the probe and the target for duplex formation to occur. The degree of stringency can be controlled by temperature, ionic strength, pH and the presence of a partially denaturing solvent such as formamide. For example, the stringency of hybridization is conveniently varied by changing the polarity of the reactant solution through manipulation of the concentration of formamide within the range of 0% to 50%.

Alternatively, the nucleic acids of interest can be amplified from nucleic acid samples using amplification techniques. For instance, PCR technology can be used to amplify the sequences of the zmet3 methyltransferase and related genes directly from genomic DNA, from cDNA, from genomic libraries or from cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes.

The degree of complementarity (sequence identity) required for detectable binding will vary in accordance with the stringency of the hybridization medium and/or wash medium. The degree of complementarity will optimally be 100 percent; however, it should be understood that minor sequence variations in the probes and primers may be compensated for by reducing the stringency of the hybridization and/or wash medium as described earlier.

Appropriate primers and probes for identifying zmet3 methyltransferase sequences from plant tissues are generated from a comparison of the sequences provided herein. For a general overview of PCR see PCR Protocols: A Guide to Methods and Applications. (Innis, M, Gelfand, D., Snisky, J. and White, T., eds), Academic Press, San Diego (1990), incorporated herein by reference.

Polynucleotides may also be synthesized by well-known techniques as described in the technical literature. See e.g., Curruthers et al., Cold Spring Harbor Symp. Quant. Biol. 47:411-418 (1982), and Adams et al., J. Am. Chem. Soc. 105:661 (1983). Double stranded DNA fragments may then be obtained either by synthesizing the complementary strand and annealing the strands together under appropriate conditions, or by adding the complementary strand using DNA polymerase with an appropriate primer sequence.

Proteins of the Present Invention

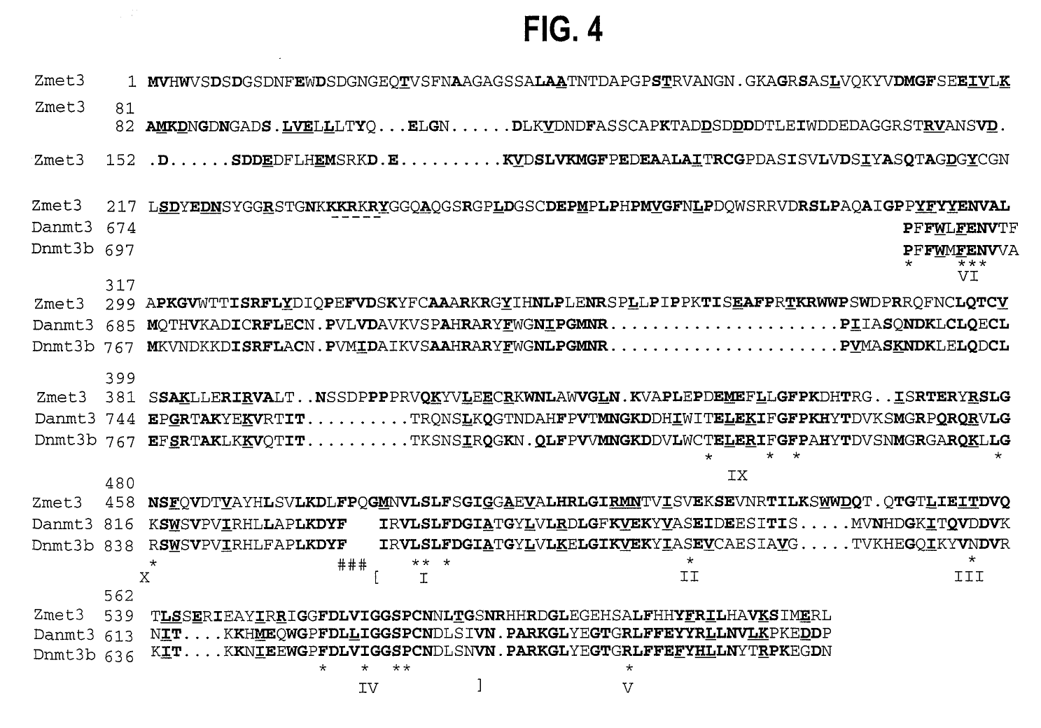

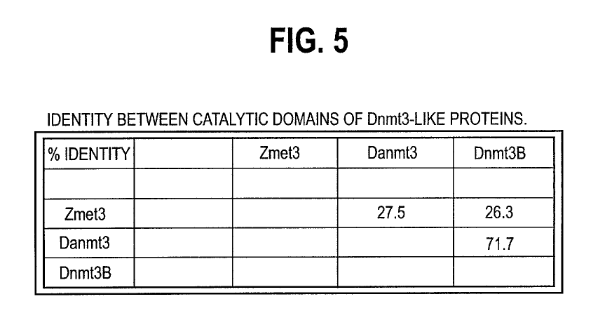

The present invention further provides for isolated zmet3 methyltransferases encoded by the zmet3 methyltransferase polynucleotides disclosed herein. One of ordinary skill in the art will recognize that the nucleic acid encoding a functional zmet3 methyltransferase need not have a sequence identical to the exemplified genes disclosed herein. For example, because of codon degeneracy, a large number of nucleic acid sequences can encode the same polypeptide. In addition, the polypeptides encoded by the zmet3 methyltransferase genes, like other proteins, have different domains which perform different functions. Specifically, zmet3 methyltransferase has conserved catalytic motifs. Most methyltransferases, including zmet3, contain these motifs from the N terminus to the C terminus of the protein. However, the zmet3 methyltransferase, unlike other eukaryotic methyltransferases, displays an altered arrangement of these motifs, specifically, VI, IX, X, I, II, III, IV, V (See FIG. 3). The location of the rearrangement can be pinpointed to a region of several amino acids between motifs X and I (See FIG. 4). It is believed that this rearrangement facilitates methylation of asymmetric sites.

Domains I and X are involved in binding AdoMet, which is source of the methyl group to be transferred during DNA methylation. Domain IV contains a catalytic domain. Domain VI aids in the positioning of domain IV. Domain VIII aids in DNA binding by neutralizing the charge of the phosphodiester backbone. The region between domain VIII and domain IX defines the sequence specificity of the zmet3 methyltransferase enzyme.

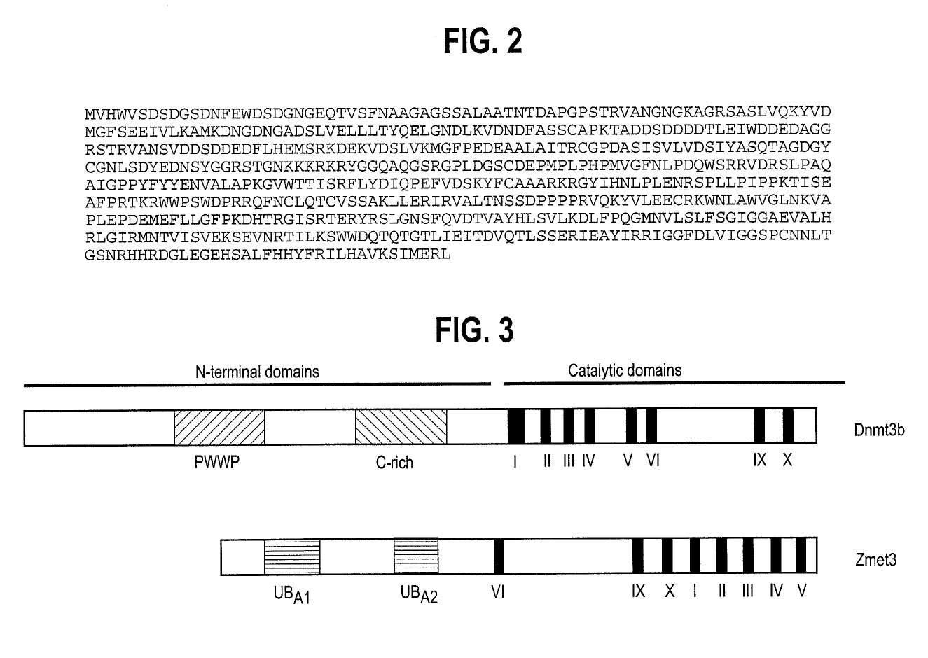

The zmet3 methyltransferase protein is at least 603 amino acid residues in length (see SEQ ID NO:2 and FIG. 2). However, those of ordinary skill in the art will appreciate that amino acid deletions, substitutions, or additions to the zmet3 methyltransferase protein will typically yield an enzyme possessing methylating characteristics similar or identical to that of the fall length sequence. Thus, full length zmet3 methyltransferase proteins modified by 1, 2, 3, 4, or 5 deletions, substitutions, or additions, generally provide an effective degree of methylation relative to the full-length protein.

Modified protein chains can also be readily designed utilizing various recombinant DNA techniques well known to those of ordinary skill in the art. For example, the chains can vary from the naturally occurring sequence at the primary structure level by amino acid substitutions, additions, deletions, and the like. Modification can also include swapping domains from the proteins of the present invention with related domains from other de novo methyltransferases.

The present invention also provides antibodies which specifically react with the zmet3 methyltransferases of the present invention under immunologically reactive conditions. An antibody immunologically reactive with a particular antigen can be generated in vivo or by recombinant methods such as by selection of libraries of recombinant antibodies in phage or similar vectors. The term “immunologically reactive conditions” as used herein, includes reference to conditions which allow an antibody, generated to a particular epitope of an antigen, to bind to that epitope to a detectably greater degree than the antibody binds to substantially all other epitopes, generally at least two times above background binding, preferably at least five times above background. Immunologically reactive conditions are dependent upon the format of the antibody binding reaction and typically are those utilized in immunoassay protocols.

The term “antibody” as used herein, includes reference to an immunoglobulin molecule obtained by in vitro or vivo generation of the humoral response, and includes both polyclonal and monoclonal antibodies. The term also includes genetically engineered forms such as chimeric antibodies (e.g., humanized murine antibodies), heteroconjugate antibodies (e.g., bispecific antibodies), and recombinant single chain Fv fragments (hereinafter “scFv”). The term “antibody” also includes antigen binding forms of antibodies (e.g., Fab′, F(ab′)₂, Fab, Fv, and, inverted IgG (See, Pierce Catalog and Handbook, (1994-1995) Pierce Chemical Co., Rockford, Ill.). An antibody immunologically reactive with a particular antigen can be generated in vivo or by recombinant methods such as selection of libraries of recombinant antibodies in phage or similar vectors (See, e.g. Huse et al., (1989) Science 246:1275-1281; and Ward, et al., (1989) Nature 341:544-546; and Vaughan et al., (1996) Nature Biotechnology, 14:309-314).

Many methods of making antibodies are known to persons of ordinary skill in the art. A number of immunogens are used to produce antibodies specifically reactive to the isolated zmet3 methyltransferase of the present invention under immunologically reactive conditions. An isolated recombinant, synthetic, or native zmet3 methyltransferase of the present invention is the preferred immunogens (antigen) for the production of monoclonal or polyclonal antibodies.

The zmet3 methyltransferase is then injected into an animal capable of producing antibodies. Either monoclonal or polyclonal antibodies can be generated for subsequent use in immunoassays to measure the presence and quantity of the zmet3 methyltransferase. Methods of producing monoclonal or polyclonal antibodies are known to those of skill in the art (See, Coligan (1991) Current Protocols in Immunology Wiley/Greene, NY; Harlow and Lane (1989) Antibodies: A Laboratory Manual Cold Spring Harbor Press, NY); and Goding (1986) Monoclonal Antibodies: Principles and Practice (2d ed.) Academic Press, New York, N.Y.).

Frequently, the zmet3 methyltransferases and antibodies will be labeled by joining, either covalently or non-covalently, a substance which provides for a detectable signal. A wide variety of labels and conjugation techniques are known and are reported extensively in both the scientific and patent literature. Suitable labels include radionucleotides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366.241.

The antibodies of the present invention can be used to screen plants for the expression of the zmet3 methyltransferases of the present invention. The antibodies of the present invention are also used for affinity chromatography in isolating zmet3 methyltransferases.

The present invention further provides zmet3 methyltransferase polypeptides that specifically bind, under immunologically reactive conditions, to an antibody generated against a defined immunogen, such as an immunogen consisting of the polypeptides of the present invention. Immunogens will generally be at least 817 contiguous amino acids from the zmet3 methyltransferase polypeptides of the present invention. Nucleic acids which encode such cross-reactive zmet3 methyltransferase polypeptides are also provided by the present invention. The zmet3 methyltransferase polypeptides can be isolated from any number of plants as discussed earlier. Preferred plants are Zea mays, Oryza sativa, Secale cereale, Triticum aestivum, Daucus carota, Brassica oleracea, Cucumis melo, Cucumis sativus, Latuca sativa, Solanum tubersoum, Lycopersicon esculentum, Phaseolus vulgaris, and Brassica napus.

As used herein, the term, “specifically binds” includes reference to the preferential association of a ligand, in whole or part, with a particular target molecule (i.e., “binding partner” or “binding moiety” relative to compositions lacking that target molecule). It is, of course, recognized that a certain degree of non-specific interaction may occur between a ligand and a non-target molecule. Nevertheless, specific binding, may be distinguished as mediated through specific recognition of the target molecule. Typically, specific binding results in a much stronger association between the ligand and the target molecule than between the ligand and non-target molecule. Specific binding by an antibody to a protein under such conditions requires an antibody that is selected for its specificity for a particular protein. The affinity constant of the antibody binding site for its cognate monovalent antigen is at least 10⁷, usually at least 10⁹, more preferably at least 10¹⁰, and most preferably at least 10¹¹ liters/mole. A variety of immunoassay formats are appropriate for selecting antibodies specifically reactive with a particular protein. For example, solid-phase ELISA immunoassays are routinely used to select monoclonal antibodies specifically reactive with a protein (See Harlow and Lane (1988) Antibodies, A Laboratory Manual, Cold Spring Harbor Publications, New York, for a description of immunoassay formats and conditions that can be used to determine specific reactivity). The antibody may be polyclonal but preferably is monoclonal. Generally, antibodies cross-reactive to zmet3 methyltransferases are removed by immunoabsorbtion.

Immunoassays in the competitive binding format are typically used for cross-reactivity determinations. For example, an immunogenic zmet3 methyltransferase polypeptide is immobilized to a solid support. Polypeptides added to the assay compete with the binding of the antisera to the immobilized antigen. The ability of the above polypeptides to compete with the binding of the antisera to the immobilized zmet3 methyltransferase polypeptide is compared to the immunogenic zmet3 methyltransferase polypeptide. The percent cross-reactivity for the above proteins is calculated, using standard calculations. Those antisera with less than 10% cross-reactivity with such proteins as zmet3 methyltransferases are selected and pooled. The cross-reacting antibodies are then removed from the pooled antisera by immunoabsorbtion with the non-zmet3 methyltransferase polypeptides.

The immunoabsorbed and pooled antisera are then used in a competitive binding immunoassay to compare a second “target” polypeptide to the immunogenic polypeptide. In order to make this comparison, the two polypeptides are each assayed at a wide range of concentrations and the amount of each polypeptide required to inhibit 50% of the binding of the antisera to the immobilized protein is determined using standard techniques. If the amount of the target polypeptide required is less than twice the amount of the immunogenic polypeptide that is required, then the target polypeptide is said to specifically bind to an antibody generated to the immunogenic protein. As a final determination of specificity, the pooled antisera is fully immunoabsorbed with the immunogenic polypeptide until no binding to the polypeptide used in the immunoabsorbtion is detectable. The fully immunoabsorbed antisera is then tested for reactivity with the test polypeptide. If no reactivity is observed, then the test polypeptide is specifically bound by the antisera elicited by the immunogenic protein.

Production of Recombinant Expression Cassettes

Isolated sequences prepared as described herein can then be used to provide recombinant expression cassettes. One of ordinary skill in the art will recognize that the nucleic acid used in the recombinant expression cassettes described herein encoding a functional zmet3 methyltransferase need not have a sequence identical to the exemplified genes disclosed herein. In addition, the polypeptides encoded by the zmet3 methyltransferase genes, like other proteins, have different domains which perform different functions. Thus, the zmet3 methyltransferase gene sequences need not be fall length, so long as the desired functional domain of the protein is expressed.

A DNA sequence coding for the desired zmet3 methyltransferase polypeptide, for example a cDNA or a genomic sequence encoding a full length protein, can be used to construct a recombinant expression cassette which can be introduced into a desired plant. An expression cassette will typically comprise the zmet3 methyltransferase polynucleotide operably linked in either the sense or antisense direction to transcriptional and translational initiation regulatory sequences which will direct the transcription of the sequence from the zmet3 methyltransferase gene in the intended tissues for the transformed plant.

For example, a plant promoter fragment may be employed which will direct expression of the zmet3 methyltransferase in all tissues of a regenerated plant. Such promoters are referred to herein as “constitutive” promoters and are active under most environmental conditions and states of development or cell differentiation. Examples of constitutive promoters includes the cauliflower mosaic virus (hereinafter “CaMV”) 35S transcription initiation region, the 1′ or 2′-promoter derived from T-DNA of Agrobacterium tumefaciens, and ubiquitin other transcription initiation regions from various plant genes known to those of ordinary skill in the art.

Alternatively, the plant promoter may direct expression of the zmet3 methyltransferase gene in a specific tissue or may be otherwise under more precise environmental or developmental control. Such promoters are referred to here as “inducible” promoters. Examples of environmental conditions that may effect transcription by inducible promoters include pathogen attack, anaerobic conditions, or the presence of light.

Examples of promoters under developmental control include promoters that initiate transcription only in certain tissues, such as leaves, roots, fruit, seeds, or flowers. The operation of a promoter may also vary depending on its location in the genome. Thus, an inducible promoter may be fully or partially constitutive in certain locations.

The endogenous promoters from the zmet3 methyltransferase genes of the present invention can be used to direct expression of the genes. These promoters can also be used to direct expression of heterologous structural genes. The promoters can be used, for example, in recombinant expression cassettes to drive expression of genes to produce DNA methyltransferase in a particular cell or tissue.

To identify the promoters, the 5 portions of the clones described herein are analyzed for sequences characteristic of promoter sequences. For instance, promoter sequence elements include the TATA box consensus sequence (TATAAT), which is usually 20 to 30 base pairs upstream of the transcription start site. In plants, further upstream from the TATA box, at positions −80 to −100, there is typically a promoter element with a series of adenines surrounding the trinucleotide G (or T) N G. J. Messing et al., in Genetic Engineering in Plants, pp. 221-227 (Kosage, Meredith and Hollaender, eds. 1983).

If proper polypeptide expression is desired, a polyadenylation region at the 3′-end of the zmet3 methyltransferase coding region should be included. The polyadenylation region can be derived from the natural gene, from a variety of other plant genes, or from T-DNA.

The vector comprising the sequences from the zmet3 methyltransferase gene will typically comprise a marker gene which confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosulforon.

As discussed above, the zmet3 methyltransferase gene can be inserted into a recombinant expression cassette in the antisense direction. Expression of the zmet3 methyltransferase gene in antisense direction will result in the production of antisense RNA. As is well known, a cell manufactures protein by transcribing the DNA of the gene encoding a protein to produce RNA, which is then processed to messenger RNA (hereinafter “mRNA”) (e.g., by the removal of introns) and finally translated by ribosomes into protein. This process may be inhibited in the cell by the presence of antisense RNA. The term “antisense RNA” means an RNA sequence which is complementary to a sequence of bases in the MRNA in question in the sense that each base (or the majority of bases) in the antisense sequence (read in the 3′ to 5′ sense) is capable of pairing with the corresponding base (G with C, A with U) in the mRNA sequence read in the 5′ to 3′ sense. It is believed that this inhibition takes place by formation of a complex between the two complementary strands of RNA, thus preventing the formation of protein. How this works is uncertain: the complex may interfere with further translation, or degrade the mRNA, or have more than one of these effects. This antisense RNA may be produced in the cell by transformation of the cell with an appropriate DNA construct designed to transcribe the non-template strand (as opposed to the template strand) of the relevant gene (or of a DNA sequence showing substantial homology therewith).

The use of antisense RNA to downregulate the expression of specific plant genes is well known. Reduction of gene expression has led to a change in the phenotype of a plant, either at the level of gross visible phenotypic difference (e.g., lack of anthocyanin production in flower petals of petunia leading to colorless instead of colored petals (see van der Krol et al., Nature, 333:866-869 (1988)), or at a more subtle biochemical level, for example, a change in the amount of polygalacturonase and reduction in depolymerization of pectin during tomato fruit ripening (Smith et al., Nature, 334:724-726 (1988)). Another more recently described method of inhibiting gene expression in transgenic plants is the use of sense RNA transcribed from an exogenous template to downregulate the expression of specific plant genes (Jorgensen, Keystone Symposium “Improved Crop and Plant Products through Biotechnology”, Abstract X1-022 (1994)). Thus, both antisense and sense RNA have been proven to be useful in achieving downregulation of gene expression in plants, which are encompassed by the present invention.

Production of Transgenic Plants

Techniques for transforming a wide variety of higher plant species using the recombinant expression cassettes hereinbefore described are well known and described in the technical and scientific literature. See, for example, Weising et al., Ann. Rev. Genet. 22:421-477 (1988).

The hereinbefore described recombinant expression cassettes may be introduced into the genome of a desired plant host by a variety of conventional techniques. For example, the DNA construct may be introduced directly into the genomic DNA of the plant cell using techniques such as electroporation, PEG poration, particle bombardment and microinjection of plant cell protoplasts or embryogenic callus, or the DNA constructs can be introduced directly to plant tissue using ballistic methods, such as DNA particle bombardment. In the alternative, the DNA constructs may be combined with suitable T-DNA flanking regions and introduced into a conventional Agrobacterium tumefaciens or Agrobacterium rhizogenes host vector. The virulence functions of the Agrobacterium host will direct the insertion of the construct and adjacent marker into the plant cell DNA when the cell is infected by the bacteria.

Transformation techniques are known in the art and well described in the scientific and patent literature. The introduction of DNA constructs using polyethylene glycol precipitation is described in Paszkowski et al., EMBO J. 3:2712-2722 (1984). Electroporation techniques are described in Fromm et al., Proc. Natl. Acad. Sci. USA 82:5824 (1985). Biolistic transformation techniques are described in Klein et al., Nature 327:70-73 (1987).

Agrobacterium tumefaciens-mediated transformation techniques are well described in the scientific literature. See, for example Horsch et al., Science 233:496-498 (1984), and Fraley et al., Proc. Natl. Acad. Sci. USA 80:4803 (1983). Although Agrobacterium is useful primarily in dicots, certain monocots can be transformed by Agrobacterium. For instance, Agrobacterium transformation of rice is described by Hiei et al., Plant J., 6:271-282 (1994).

Transformed plant cells which are derived by any of the above transformation techniques can be cultured to regenerate a whole plant which possesses the transformed genotype. Such regeneration techniques rely on manipulation of certain phytohormones in a tissue culture growth medium, typically relying on a biocide and/or herbicide marker which has been introduced together with the zmet3 methyltransferase nucleotide sequences. Plant regeneration from cultured protoplasts is described in Evans et al., Protoplasts Isolation and Culture, Handbook of Plant Cell Culture, pp. 124-176, MacMillian Publishing Company, New York, 1983; and Binding; Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al., Ann. Ref of Plant Phys. 38:467-486 (1987).

The methods of the present invention are particularly useful for incorporating the zmet3 methyltransferase polynucleotides into transformed plants in ways and under circumstances which are not found naturally. In particular, the zmet3 methyltransferase may be expressed at times or in quantities which are not characteristic of natural plants.

One of ordinary skill in the art will recognize that after the expression cassette is stably incorporated in transgenic plants and confirmed to be operable, it can be introduced into other plants by sexual crossing. Any of a number of standard breeding techniques can be used, depending upon the species to be crossed.

The hereinbefore described expression cassettes can be inserted into a plant in order to reduce or alter the amount of DNA methylation in a plant. Preferably, such an expression cassette contains the zmet3 methyltransferase gene inserted into the cassette in the antisense direction as described earlier. A reduction or alteration in the amount of DNA methylation in a plant can be used to stabilize transgene expression in a transgenic plant.

One of the difficulties with the production of transgenic plants is that many transgenes are silenced or are not stable through successive generations. In many cases, transgene silencing is associated with increased DNA methylation. The hereinbefore described expression cassettes of the present invention containing the zmet3 methyltransferase gene in the antisense direction can be inserted into a plant either before, concurrently with or after the insertion of another expression cassette containing a transgene which is to be expressed in the plant, such as, but not limited to, a resistance or drought tolerance gene, etc. The antisense RNA produced by the hereinbefore described expression cassette can then form a complex with the endogenous mRNA from the zmet3 methyltransferase gene within the plant. This complex should reduce or alter the amount of DNA methylation occurring in vivo in the plant. This reduction in DNA methylation should prevent the silencing of the desired transgene in the plant.

In a similar manner, the expression cassettes described herein can be used to modify or alter the yield or biochemical qualities of a plant. As discussed earlier, certain genes in plants and animals are expressed differentially when transmitted thorough a male versus female parent. This phenomenon is known as imprinting. Imprinting is an epigenetic system correlated with DNA methylation. A reduction or alteration of DNA methylation in a plant by transforming a plant with an expression cassette containing the zmet3 methyltransferase gene in the antisense direction may affect the yield and biochemical qualities of a plant.

The hereinbefore described expression cassettes can also be used to silence the expression of a particular targeted gene in plants in vivo. More specifically, the expression cassettes of the present invention containing a tissue-specific promoter and the zmet3 methyltransferase gene in the sense direction can be inserted into a plant. The tissue-specific promoter will direct expression of the zmet3 methyltransferase gene in a area containing the desired targeted gene. Translation of the zmet3 methyltransferase gene in the specific area will result in an increase in methylation in the area of the targeted gene. This increase in methylation can silence the targeted gene.

Transgenic plants containing the expression cassettes described herein and which exhibit a reduction in DNA methylation can be identified by using methylation sensitive restriction enzymes or High Performance Liquid Chromatography. Techniques for using methylation sensitive restriction enzymes and High Performance Liquid Chromatography are well known in the art. Transgenic plants containing the expression cassettes described herein and which exhibit an increase in DNA methylation can be identified by using a Northern Blot analysis which is well known in the art.

Additionally, the hereinbefore described expression cassettes can be used in gene therapy for human diseases which are caused by the amplification of trinucleotide repeats.

The following Examples are offered by way of illustration, not limitation.

### EXAMPLE 1

### Isolation and characterization of zmet3.

cDNA cloning and RACE analysis.

The maize Dnmt3-like sequence was found by searching a collection of Expressed Tag Sequences (hereinafter “ESTs”) at Pioneer Hi-Bred International Inc. (Des Moines, Iowa), for sequences similar to mouse Dnmt3 (see Okano, M., et al., Nature Genetics, 19:219-220 (1998), herein incorporated by reference). All of the ESTs appeared to correspond to an identical sequence, which was named zmet3. To clone the full-length zmet3 cDNA sequence, 5′ Rapid amplification of cDNA ends (hereinafter “RACE”) PCR was performed on Marathon cDNA (Clontech) using Advantage2 DNA polymerase (Clontech). The primers used for RACE were Dmt3F1 (5′- ATCCGTATGCCAAGCCTGTGGAGAGC-3′) (SEQ ID NO:3), Dmt3F2 (GATGGACTTGACGGCGTGTAAGATCC-3′) (SEQ ID NO:4), Zmet3RACE1 (5′-GGAGGAAGTGGCAGAGGAGGAGG-3′) (SEQ ID NO:5) and Zmet3RACE2 (5′- GGAGGCACTGGACGGCGTGG-3′) (SEQ ID NO:6). RACE products were directly sequenced and cloned into pGEM-T Easy (Promega).

Genomic Southern Blots.

Maize genomic DNA was isolated from T×303 and Cm37 (each available from the Germplasm Repository, North Central Regional Plant Introduction Station—USDARS and Iowa State University, Ames, Iowa) leaf tissue. 8 ug of DNA was digested and electrophoresed in 0.9% agarose gels and transferred onto Hybond-N (Amersham) membranes. 50 ng of the 5′ 1755 base pair of the zmet3 cDNA sequence were random prime labeled with ³²p Washes were performed at high stringency; 0.1×SSC, 0.5% SDS for 30 minutes at 60° C., and 0.1×SSC, 0.1% SDS for 30 minutes at 60° C.

RNA Blot Analysis and RTPCR.

Total RNA was extracted from tissues including embryo, leaf, immature ear, immature tassel, 3-day-old root, pollen and Black Mexican (available from the Germplasm Repository, North Central Regional Plant Introduction Station—USDARS and Iowa State University, Ames, Iowa) suspension cultures using TRIzol (Life Technologies Gibco/BRL). PolyA+ RNA, isolated using PolyAtract (Promega) was used to make cDNA with a Marathon cDNA Amplification Kit (Clontech). 2ng of cDNA was used in each PCR reaction. The primers used were: Dmt3F1 (5′- ATCCGTATGCCAAGCCTGTGGAGAGC-3′) (SEQ ID NO:3), Dmt3F2 (GATGGACTTGACGGCGTGTAAGATCC-3′) (SEQ ID NO:4), Zmet3RACE1 (5′-GGAGGAAGTGGCAGAGGAGGAGG-3′) (SEQ ID NO:5), Dmt3R1 (5′- GGC TTT CCG AAG ATC GAC ACG AGA GG-3′) (SEQ ID NO:7) and Dmt3R2 (5′- TCA GTG GAG AAG TCC GAG GTC AAC C-3′) (SEQ ID NO:8).

Results.

To examine the relationships between the zmet3 gene and other known methyltransferases, alignments were performed using the conserved catalytic motifs I-IV (FIG. 4). Representatives of four classes of animal and plant DNA methyltransferases were used in the alignments, including enzymes of the Dnmt1/MET1 maintenance methyltransferase class, as well as the Dnmt2, CMT, and Dnmt3 classes. Zmet3 and the related soybean EST sequence group with a 99% bootstrap value to the clade containing the de novo methyltransferase proteins Dnmt3a and Dnmt3b from mammals and zebrafish (Danio rerio).

Consistent with its putative function as a DNA methyltransferase, the zmet3 protein is predicted by PSORT (Nakai, K., et al., Genomics 14:897-911 (1992)) to reside in the nucleus and contain conserved nuclear targeting sequences of the SV40 large T antigen type. This lies in the N terminus of the protein (underlined in FIG. 3). The Dnmt3 methyltransferases contain two recognizable protein motifs in their N termini, a PWWP domain of unknown function and a cysteine-rich region that shows homology to the X-linked A TRX gene of the SNF2/SW1 family (Xie, S., et al., Gene 236:87-95 (1999); Xu, G. L., et al., Nature 402:187-191 (1999)). Zmet3 does not appear to contain such domains.

To determine if Zmet3 contained any recognizable domains in their N termini, the protein sequence was tested on both the PFAM and SMART (Schultz, J., et al., Proc. Natl. Acad. Sci. USA 95:5857-64 (1998)) protein prediction web servers. Both programs predicted two UBA domains in Zmet3 (FIG. 3). UBA domains are found in several ubiquitination pathway enzymes, in proteins involved in nucleotide excision repair (such as Rad23), and in some protein kinases (Hofmann, K., et al., Trends Biochem. Sci. 21:172-173 (1996)). The NMR structure of a UBA domain from the human homolog of Rad23 (HHR23A) shows that it folds into a compact three helix bundle (Dieckmann, T., et al., Nat. Struct. Biol. 5:1042-1047 (1998)).

To assay the complexity of the gene families encoding the Zmet3 type protein, Southern blot analysis was performed. Southern blot analysis using a ZMET3 probe detected several hybridizing bands suggesting the presence of a small gene family of ZMET3-like genes. A blast search of GenBank using the full-length Zmet3 sequence detected a maize EST sequence encoding a related protein (accession AI947339). However, as this sequence lacks a highly conserved PC site in motif IV of the catalytic domain, and hence is likely to be a pseudogene.

Reverse transcription-polymerase chain reaction (hereinafter “RT-PCR”) was used to study the expression of zmet3 in different tissues. Roughly similar amounts of PCR products were detected from RNA of embryos, roots, leaves, immature tassels, immature ears and callus tissue.

Discussion of Results.

The polynucleotide sequence of zmet3 contains a novel arrangement of the conserved catalytic motifs. Most methyltransferases contain motifs I, II, III, IV, V, VI, IX, X from the N terminus to the C terminus of the protein. However zmet3 displays an altered arrangement of these motifs, specifically, VI, IX, X, I, II, III, IV, V. The location of the rearrangement can be pinpointed to a region of several amino acids between motifs X and I. While not wishing to be bound by any theory, the inventors believe that there are at least two processes that could have given rise to the rearrangement of the conserved motifs. The first is a transposition even resulting in a swap between motifs I-V and motifs VI-X. The second possibility is gene duplication followed by deletions to remove motifs I-V of the first gene, the intervening sequence between the two genes, and motifs VI-X of the second gene. Zmet3 is the first example of a eukaryotic gene displaying a rearranged DNA methyltransferase motif.

Given the relationship of the plant genes to Dnmt3, the inventors believe that Zmet3 acts as plant de novo methyltransferases. Several well-characterized examples of de novo methylation occur in plants. One case is the extensive methylation at the SUPERMAN locus in the Arabidopsis clark kent mutants and in plants containing antisense-MET1 constructs (Jacobsen, S. E., et al., Science 277:1100-1103 (1997)).

### EXAMPLE 2

### Zmet3 plants containing a mutator gene have small leaves with little to no blade and do not survive to maturity

A reverse genetics approach was used to ascertain the function of zmet3. A F₂ family segregating for a Mutator (Mu) insertion was identified using a PCR primer for Mu and a gene-specific primer for zmet3. This allele is called zmet3-E03. The insertion is in an intron 5′ of base pair 265 in the zmet3 cDNA sequence (FIG. 1). The molecular consequence of this insertion has not been determined, but the segregation data described below indicates that the insertion affects gene function. The most likely explanation for altered gene function with an intron insertion is imprecise splicing, although other mechanisms such as disruption of enhancer sequences, or nucleating silencing chromatin are also possible.

Fourteen (14) plants segregating for the zmet3-E03 insertion were grown in glasshouses in St. Paul, Minn. and at the West Madison research station in Madison, Wis. in 2000. Plants within families segregating for the zmet3-E03 insertion exhibit a phenotype of small leaves with little to no blade and do not survive to maturity. More specifically, eight of these plants were grown in St. Paul, Minn. and six of these plants were grown in Madison, Wis. Three of the eight plants grown in St. Paul, Minn. exhibited the aberrant phenotype and were found to contain at least one copy of the zmet3-E03 allele, although the inventors were unable to determine whether or not these plants were homozygous for this allele. Two of the six plants grown in Madison, Wis. exhibited the aberrant phenotype. These plants were found to be homozygous for the zmet3-E03 allele. While not wishing to be bound by any theory, the inventors believe that this data suggests that zmet3 is required for normal maize development and that disruption of the function of these gene will alter normal development and will prevent plants from maturing normally.

The present invention is illustrated by way of the foregoing description and examples. The foregoing description is intended as a non-limiting illustration, since many variations will become apparent to those skilled in the art in view thereof. It is intended that all such variations within the scope and spirit of the appended claims be embraced thereby.

Changes can be made to the composition, operation and arrangement of the method of the present invention described herein without departing from the concept and scope of the invention as defined in the following claims.

What is claimed is:

1. An isolated and purified Zea mays zmet3 methyltransferase polynucleotide.

2. The polynucleotide sequence of claim 1 wherein the polynucleotide hybridizes to SEQ ID NO:1 under stringent conditions.

3. A zmet3 methyltransferase comprising the amino acid sequence shown in SEQ ID NO:2.

4. An expression cassette comprising a promoter sequence operably linked to the isolated and purified polynucleotide of claim 1.

5. The expression cassette of claim 4 further comprising a polyadenylation signal operably linked to the polynucleotide.

6. The expression cassette of claim 4 wherein the promoter is a constitutive or tissue specific promoter.

7. The expression cassette of claim 4 wherein the polynucleotide hybridizes to SEQ ID NO:1 under stringent conditions.

8. A bacterial cell comprising the expression cassette of claim 4.

9. The bacterial cell of claim 8 wherein the bacterial cell is an Agrobacterium tumefaciens cell or an Agrobacterium rhizogenes cell.

10. A plant cell transformed with the expression cassette of claim 4.

11. A transformed plant containing the plant cell of claim 10.

12. The transformed plant of claim 11 wherein the plant is Zea mays, Oryza sativa, Secale cereale, Triticum aestivum, Daucus carota, Brassica oleracea, Cucumis melo, Cucumis sativus, Latica sativa, Solanum tubersoum, Lycopersicon esculentum, Phaseolus vulgaris, and Brassica napus.

13. Seed from the transformed plant of claim 11.

14. Transformed plant seed containing the plant cell of claim 8.

15. A process for methylating a target gene in a plant, the process comprising the steps of:
transforming a plant with a recombinant expression cassette comprising a tissue specific promoter and the polynucleotide of claim 1, the tissue specific promoter being operably linked to the polynucleotide, wherein the tissue-specific promoter directs expression of the polynucleotide, and the expression of the polynucleotide produces zmet3 methyltransferase in sufficient quantities in the are containing the target gene to allow for methylation of the target gene.

16. The process of claim 15 wherein the plant is Zea mays, Oryza sativa, Secale cereale, Triticum aestivum, Daucus carota, Brassica oleracea, Cucumis melo, Cucumis sativus, Latuca sativa, Solanum tubersoum, Lycopersicon esculentum, Phaseolus vulgaris, and Brassica napus.

17. The process of claim 15 wherein the polynucleotide hybridizes to SEQ ID NO:1 under stringent conditions.
